(19) United States
(12) Patent Application Publication
(10) Pub. No.: US 2004/0034058 A1
(43) Pub. Date: Feb. 19, 2004

(54) Novel pharmaceutical formulation suitable for nebulisation

(76) Inventors: Duncan Armour, Sandwich (GB); David Brown, Welwyn Garden City (GB); Miles Congreve, Cambridge (GB); Paul Gore, Stevenage (GB); Darren Green, Stevenage (GB); Stuart Holman, Stevenage (GB); Torquil Jack, Stevenage (GB); Andrew Mason, Stevenage (GB); Karen Morriss, Stevenage (GB); Nigel Ramsden, Stevenage (GB); Marian Thomas, Ware (GB); Peter Ward, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/311,561

(22) PCT Filed: Jun. 15, 2001
(86) PCT No.: PCT/GB01/02688

(51) Int. Cl. A61K 31/445

(52) U.S. Cl. 514/317; 514/330

(57) ABSTRACT

The present invention relates to a pharmaceutical formulation suitable for nebulisation which comprises an aqueous solution of (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof. Methods and uses of the formulation in the treatment of respiratory disorders such as asthma are also described.

The present invention relates to a pharmaceutical formulation for use in the administration of medicaments by inhalation. In particular, this invention relates to a pharmaceutical formulation for use in nebulisers. The invention also relates to methods for their preparation and to their use in therapy.

(2S)-3-[4-({[4-(Aminocarbonyl)-1-piperidinyl]carbonyl}oxy)phenyl]-2-[((2S)-4-methyl-2{[2-(2-methylphenoxy)acetyl]amino}pentanoyl)amino] propanoic acid has recently been disclosed in International Patent Application (PCT/EP99/10000) as a novel antagonist of both α4β1 and α4β7 integrins which, as a consequence, results in effective anti-inflammatory properties.

Anti-inflammatory conditions, eg. asthma are typically treated by medicaments in the form of dry powders or aerosols containing small particles of the medicament, conventionally prepared by micronisation. Generally, these medicaments are administered by means of metered dose inhalers, which are designed to deliver a fixed unit dosage of medicament per actuation or “puff”. However, some patients, in particular children and the elderly, have difficulty in coordinating actuation of a metered dose inhaler with inhalation, and are therefore unable to use this mode of administration effectively. Furthermore, a proportion of patients find inhalation of dry powders difficult or unpleasant. There is therefore a demand for a pharmaceutical formulation containing anti-inflammatory medicaments in a form suitable for nebulisation.

Thus, according to the present invention we provide a pharmaceutical formulation suitable for nebulisation which comprises:

an aqueous solution of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof.

Preferably, the formulation will contain one or more buffer agents.

Preferably, the formulation will contain one or more isotonicity adjusting agents.

According to one particular aspect of the present invention we provide a pharmaceutical formulation which comprises:

(i) an aqueous solution of (2S)-3-[4-({[4-(Aminocarbonyl)-1-piperidinyl]carbonyl}oxy)phenyl]-2-[((2S)-4-methyl-2{[2-(2-methylphenoxy)acetyl]amino}pentanoyl)amino] propanoic acid or a salt or solvate thereof;

(ii) one or more buffer agents; and

(iii) one or more isotonicity adjusting agents.

Examples of suitable salts include physiologically acceptable salts such as alkali metal salts, for example calcium, sodium and potassium salts and salts with (trishydroxymethyl)aminomethane.

Preferably the (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid is present as the potassium salt.

For introduction of the (2S)-3-[4-({[4-(Aminocarbonyl)-1-piperidinyl]carbonyl}oxy) phenyl]-2-[((2S)-4-methyl-2-{[2-(2-methylphenoxy)acetyl]amino}pentanoyl)amino] propanoic acid (or a salt or solvate thereof) into the lungs, the droplet size of the nebulised formulation is an important parameter. Droplet size depends to some extent on the type of nebuliser used, whether a facemask or a mouthpiece is used and, for jet nebulisers, the pressure or flow rate of the compressed gas, as well as on the physical properties of the formulation for nebulisation. The nebulised formulation will be heterodisperse, i.e. droplets will cover a range of sizes. Typically, mean droplet size will be in the range of 1 to 15 microns, preferably 1 to 10 microns, more preferably less than 7 microns.

The formulation according to the invention desirably contains 1 to 50 mg of (2S)-3-[4-({[4-(Aminocarbonyl)-1-piperidinyl]carbonyl}oxy)phenyl]-2-[((2S)-4-methyl-2{[2-(2-methylphenoxy)acetyl]amino}pentanoyl)amino] propanoic acid (or a salt or solvate thereof) per 2 ml dose.

The formulations according to the invention are buffered to a pH of from about 5 to about 7, preferably about 6. Suitable buffers are those which are physiologically acceptable upon administration by inhalation. Such buffers include citric acid buffers and phosphate buffers, of which phosphate buffers are preferred. Particularly preferred buffers for use in the formulations of the invention are monosodium phosphate dihydrate and dibasic sodium phosphate anhydrous.

The aqueous component is preferably a high grade quality of water, most preferably purified water.

The formulations according to the invention will desirably be isotonic with the fluids of the lung. The formulations may be adjusted to isotonicity by addition of a suitable salt, for example, sodium chloride, dextrose or calcium chloride.

Thus, in a preferred embodiment, the formulations according to the invention additionally comprise sufficient sodium chloride, or another suitable pharmaceutically acceptable salt, to provide an isotonic composition.

In a particularly preferred embodiment, the invention provides a formulation suitable for administration by nebulisation, which formulation consists of:

(a) (2S)-3-[4-({[4-(Aminocarbonyl)-1-piperidinyl]carbonyl}oxy) phenyl]-2-[((2S)-4-methyl-2-{[2-(2-methylphenoxy)acetyl]amino}pentanoyl)amino] propanoic acid (or a salt or solvate thereof) 0.1-20 mg;

(b) monosodium phosphate dihydrate 18-20 mg;

(c) dibasic sodium phosphate anhydrous 3-4 mg;

(d) sodium chloride 9-10 mg; and

(e) water for injection to 2.0 ml

Optionally a further active ingredient suitable for inhalation therapy may be incorporated into the formulation such as a corticosteroid (eg fluticasone propionate) or a bronchodilator (eg salmeterol or albuterol or a salt thereof).

The chemical and physical stability and the pharmaceutical acceptability of the formulations according to the invention may be determined by techniques well known to those skilled in the art. Thus, for example, the chemical stability of the components may be determined by HPLC assay, for example, after prolonged storage of the product.

The formulations according to the invention may be prepared by conventional methods for the preparation of nebuliser formulations. Typically the formulation is sterilised, conveniently by means of thermal sterilisation using steam. Aliquots of the formulation are conveniently filled into sterile containers, for example unit dose containers such as vials or ampoules which are suitably moulded from thermoplastics.

Examples of disease states in which the formulation of the present invention has potentially beneficial anti-inflammatory effects include respiratory disorders, more particularly asthma.

Thus, according to a further aspect of the invention we provide a pharmaceutical formulation of the present invention for use in the treatment or prophylaxis of respiratory disorders such as asthma by inhalation.

We also provide a use of a pharmaceutical formulation of the present invention in the manufacture of a medicament for the treatment or prophylaxis of respiratory disorders such as asthma by inhalation.

We also provide a method of treatment of respiratory disorders such as asthma which comprises administering to a patient by inhalation a pharmaceutically acceptable amount of the formulation of the present invention.

The invention is further illustrated by the following non-limiting examples:

### EXAMPLE A

### (2S)-3-[4-({[4-(Aminocarbonyl)-1-piperidinyl]carbonyl} oxy) phenyl]-2-[((2S)-4-methyl-2{[2-(2-methylphenoxy)acetyl]amino}pentanoyl)amino] Propanoic Acid Potassium Salt

To Wang resin (50 g) was added a solution of (2S)-3-[4-(allyloxy)phenyl]-2-[(tert-butoxycarbonyl)amino]propanoic acid (115.8 g) and 1-hydroxybenzotriazole (48.6 g) in DMF (475 ml). After 15 minutes 1,3-diisopropylcarbodiimide (56.5 ml) was added and the mixture was stirred for 24 h at 45° C. The resin was filtered and washed with DMF (3×360 ml), methanol (3×360 ml) and dichloromethane (3×700 ml). To a slurry of the resin in dichloromethane (644 ml) was added pyridine (14.7 ml). Acetic anhydride (26.9 ml) was added and the mixture was stirred for 12 h at 20° C. The resin was filtered and washed with dichloromethane (3×550 ml), methanol (3×370 ml) and dichloromethane (3×550 ml).

A slurry of 20 g of the resin in dichloromethane (100 ml) was cooled to 2-5° C. and treated with a solution of phenol (20 g) in dichloromethane (80 ml). Chlorotrimethylsilane (20 ml) was added dropwise and the mixture was stirred for 6 h at 2-5° C. The resin was filtered and washed with dichloromethane (3×200 ml), methanol (3×200 ml), 10% water in DMF (2×200 ml), 10% diisopropylethylamine in DMF (3×200 ml), DMF (200 ml), methanol (3×200 ml) and dichloromethane (3×200 ml).

A slurry of the resin in DMF (55 ml) was treated with a solution of Fmoc-leucine (32.7 g) and 1-hydroxybenzotriazole (12.5 g) in DMF (85 ml). After 5 minutes 1,3-diisopropylcarbodiimide (19.3 ml) was added and the mixture was stirred for 15 h at 20° C. The resin was filtered and washed with DMF (3×150 ml), methanol (3×150 ml) and dichloromethane (3×150 ml).

The resin was treated with 20% piperidine in DMF (180 ml) and stirred for 1 h at 20° C. The resin was filtered and washed with DMF (3×150 ml), dichloromethane (3×150 ml), DMF (3×150 ml) and dichloromethane (3×150 ml). To a slurry of this in DMF (50 ml) was added a solution of (2-methylphenoxy)acetic acid (17.9 g) and 1-hydroxybenzotriazole (14.6 g) in DMF (100 ml). After 5 minutes 1,3-diisopropylcarbodiimide (16.9 ml) was added and the mixture was stirred for 65 h at 20° C. The resin was filtered and washed with DMF (2×150 ml), methanol (3×150 ml) and dichloromethane (3×150 ml).

A slurry of the resin in dichloromethane (60 ml) was treated with a solution of tetrakis(triphenylphosphine)palladium(0) (5.21 g) in dichloromethane (140 ml) followed by morpholine (13 ml). The mixture was stirred for 2 h at 20° C. then the resin was filtered and washed with dichloromethane (7×200 ml).

A slurry of the resin in dichloromethane (160 ml) was treated with diisopropylethylamine (12.4 ml) followed by 4-nitrophenyl chloroformate (24.8 g) in 3 portions at 5 minute intervals. The mixture was stirred for 1 h at 20° C. The resin was filtered and washed with dichloromethane (3×200 ml). The resin was treated with a solution of isonipecotamide (15.8 g) in DMF (180 ml) and the mixture was stirred for 1.5 h at 20° C. The resin was filtered and washed with DMF (4×200 ml) and dichloromethane (2×200 ml).

The resin was treated with 50% TFA in dichloromethane (200 ml). After stirring for 1 h at 20° C. the resin was filtered and washed with dichloromethane (5×200 ml). The combined filtrate and washings were evaporated in vacuo. The residue was azeotroped with toluene (2×100 ml) then triturated with ether (50 ml) and the resulting white solid filtered. To this was added acetonitrile (150 ml) and the mixture was heated to reflux. The resulting suspension was allowed to cool to 20° C. and stirred for 18 h. The mixture was filtered to give (2S)-3-[4-({[4-(Aminocarbonyl)-1-piperidinyl]carbonyl} oxy) phenyl]-2-[((2S)-4-methyl-2-{[2-(2-methylphenoxy)acetyl]amino}pentanoyl) amino] propanoic acid as a white solid (4.9 g).

A suspension of (2S)-3-[4-({[4-(Aminocarbonyl)-1-piperidinyl]carbonyl} oxy) phenyl]-2-[((2S)-4-methyl-2-{2-(2-methylphenoxy) acetyl]amino}pentanoyl) amino] propanoic acid (10 g) in methanol (150 ml) was warmed to reflux to obtain a clear solution. To this was added a solution of potassium carbonate (1.16 g) in water (7.5 ml). After heating under reflux for two minutes the solvents were evaporated in vacuo to give a crisp foam. To this was added acetonitrile (100 ml) and the mixture was warmed to reflux, during which time the foam collapsed and started to crystallise. After ten minutes the mixture was allowed to cool to 20° C. then filtered under reduced pressure, washed with acetonitrile (25 ml) and ether (50 ml) to give the title compound as a white solid (10.65 g, 100%).

| | mg |
|---|---|
| Example 1 | |
| (2S)-3-[4-({[4-(Aminocarbonyl)-1-piperidinyl]carbonyl}oxy) | 20 |
| phenyl]-2-[((2S)-4-methyl-2-{[2-(2- | |
| methylphenoxy)acetyl]amino}pentanoyl)amino] propanoic | |
| acid potassium salt (prepared according to Example A) | |
| Monosodium phosphate dihydrate | 18.80 |
| Dibasic sodium phosphate anhydrous | 3.50 |
| Sodium chloride | 9.60 |
| Water for injection (Stilmas) | to 2.00 ml |
| Example 2 | |
| (2S)-3-[4-({[4-(Aminocarbonyl)-1-piperidinyl]carbonyl}oxy) | 50 |
| phenyl]-2-[((2S)-4-methyl-2-{[2-(2- | |
| methylphenoxy)acetyl]amino}pentanoyl)amino] propanoic | |
| acid potassium salt (prepared according to Example A) | |
| Monosodium phosphate dihydrate | 18.80 |
| Dibasic sodium phosphate anhydrous | 3.50 |
| Sodium chloride | 9.60 |
| Water for injection (Stilmas) | to 2.00 ml |

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

Above mentioned patent applications are hereinbefore incorporated by reference.

1. A pharmaceutical formulation suitable for nebulisation which comprises:
an aqueous solution of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof.

2. A formulation according to claim 1 which comprises one or more buffer agents.

3. A formulation according to claim 2 which is buffered to a pH of from about 5 to about 7.

4. A formulation according to claim 3 which is buffered to a pH of about 6.

5. A formulation according to any one of claims 2 to 4 wherein the buffers include citric acid buffers and phosphate buffers.

6. A formulation according to claim 5 wherein the buffers are phosphate buffers.

7. A formulation according to claim 6 wherein the buffers are monosodium phosphate dihydrate and dibasic sodium phosphate anhydrous.

8. A formulation according to any one of claims 1 to 7 which comprises one or more isotonicity adjusting agents.

9. A formulation according to claim 8 wherein the isotonicity adjusting agent is selected from sodium chloride, dextrose or calcium chloride.

10. A formulation according to claim 9 wherein the isotonicity adjusting agent is sodium chloride.

11. A formulation according to any one of claims 1 to 10 which comprises water.

12. A formulation according to any one of claims 1 to 11 which comprises:
(i) an aqueous solution of (2S)-3-[4-({[4-(Aminocarbonyl)-1-piperidinyl]carbonyl}oxy)phenyl]-2-[((2S)-4-methyl-2{[2-(2-methylphenoxy)acetyl]amino}pentanoyl)amino] propanoic acid or a salt or solvate thereof;
(ii) one or more buffer agents;
(iii) one or more isotonicity adjusting agents; and

13. A formulation according to any one of claims 1 to 12 wherein the (2S)-3-[4-({[4-(Aminocarbonyl)-1-piperidinyl]carbonyl}oxy)phenyl]-2-[((2S)-4-methyl-2{[2-(2-methylphenoxy)acetyl]amino}pentanoyl)amino] propanoic acid is present as the potassium salt.

14. A formulation according to any one of claims 1 to 13 which contains 1 to 50 mg of (2S)-3-[4-({[4-(Aminocarbonyl)-1-piperidinyl]carbonyl}oxy) phenyl]-2-[((2S)-4-methyl-2-{[2-(2-methylphenoxy)acetyl]amino}pentanoyl)amino] propanoic acid (or a salt or solvate thereof) per 2 ml dose.

15. A formulation suitable for administration by nebulisation, which formulation consists of:
(a) (2S)-3-[4-({[4-(Aminocarbonyl)-1-piperidinyl]carbonyl}oxy) phenyl]-2-[((2S)-4-methyl-2-{[2-(2-methylphenoxy)acetyl]amino}pentanoyl)amino] propanoic acid (or a salt or solvate thereof) 0.1-20 mg;
(b) monosodium phosphate dihydrate 18-20 mg;
(c) dibasic sodium phosphate anhydrous 3-4 mg;
(d) sodium chloride 9-10 mg; and
(e) water for injection to 2.0 ml

16. A pharmaceutical formulation according to any one of claims 1 to 15 for use in the treatment or prophylaxis of respiratory disorders by inhalation.

17. Use of a pharmaceutical formulation according to any one of claims 1 to 15 in the manufacture of a medicament for the treatment or prophylaxis of respiratory disorders by inhalation.

18. A method of treatment of respiratory disorders which comprises administering to a patient by inhalation a pharmaceutically acceptable amount of the formulation according to any one of claims 1 to 15.
